(19) United States
(12) Patent Application Publication
(10) Pub. No.: US 2002/0136886 A1
(43) Pub. Date: Sep. 26, 2002

(54) Porous wick for liquid vaporizers

(76) Inventors: Mengtao Pete He, Scottsdale, AZ (US); Carl Triplett, Scottsdale, AZ (US); Debra Park, Mesa, AZ (US); Christopher J. Wolpert, Scottsdale, AZ (US); Kristopher J. Stathakis, Scottsdale, AZ (US)

Correspondence Address: Damon L. Boyd, Snell & Wilmer L.L.P., 400 East Van Buren, Phoenix, AZ, 85004-2202, US

(21) Appl. No.: 09/974,634

(22) Filed: Oct. 9, 2001

Related U.S. Application Data
(60) Provisional application No. 60/239,621, filed on Oct. 9, 2000.

(51) Int. Cl. A61L 9/04; A24F 25/00; B32B 3/26; B32B 3/00

(52) U.S. Cl. 428/313.5; 428/304.4; 428/313.3; 428/315.7; 239/44

(57) ABSTRACT

The present invention relates to the use of porous materials, for transporting liquids from a reservoir in a vapor dispensing device which addresses many of the shortcomings of the prior art, by selecting pore sizes and void volume ratios of the various wicking materials to fall within an effective range to obtain effective control of liquid delivery. For example, in accordance various aspects of the present invention, a porous polymeric wick is comprised of various polymeric materials having pore sizes less than about 250 microns and void volume ratios from about 25 to about 60%.

### CROSS REFERENCE TO RELATED APPLICATION

This United States Utility Patent Application claims priority to U.S. Provisional Patent Application Serial No.60/239,621 filed Oct. 9, 2000.

### FIELD OF THE INVENTION

This invention generally relates to vapor-dispensing devices and more particularly to porous wicks having improved vapor dispensing capabilities.

### BACKGROUND OF THE INVENTION

There have been various methods devised to attempt to regulate the diffusion of volatile materials especially with regard to the vapor delivery of fragrances and/or deodorizers. Exemplary prior art devices which relate to this are U.S. Pat. Nos.: 525,646; 1,123,036; 1,129,897; 1,323,659; 1,377,909; 2,383,960; 2,507,889; 2,616,759; 2,657,090; 2,787,496; 2,797,844; 2,878,060; 2,961,167; 2,975,464; 3,104,816; 3,239,145; 3,550,853; 3,633,881; 3,679,133; 3,804,331; 4,014,501; 4,094,639; 4,413,779; 4,663,315; 4,739,928; 5,038,394; 5,647,053; 5,903,710; 5,945,094; 5,976,503; and 6,104,867. The primary function of these types of devices has generally been the counteracting of malodors as well as the delivery of aesthetically pleasing fragrance vapors or other vaporizable materials. Liquid air fresheners and other vapor-dispensing products currently on the market typically have a fluid-reservoir and a transport system from which the fluid is evaporated and/or dispensed into the surrounding air.

One approach to dispensing fluids, fragrances for example, has been to drip the fragrance liquid from the reservoir onto a porous substrate of relatively large surface area where the fragrance is evaporated from the substrate surface. Another method has been to partially immerse a wick made of porous material in a liquid fragrance-reservoir where the liquid is transported through the wick by capillary action. The fragrance is then evaporated from the exterior wick surface into the surrounding air.

If the space proximal to the wick is heated by an electrical heating element, the fragrance delivery device is often referred to as an electric liquid air freshener. In such devices, the heating element delivers kinetic energy to molecules of the fragrance solution on the exterior surface of the wick thereby increasing the rate of evaporation to obtain higher fragrance intensity and uniform delivery density over time.

Products currently on the market have utilized wicks constructed of compressed graphite, porous ceramic, or fibrous bundles. See, for example, U.S. Pat. No. 4,663,315 issued May 5, 1987 to Hasegawa et al. and U.S. Pat. No. 4,739,928 issued Apr. 26, 1988 to O'Neil. With such wicks, the transport mechanism is capillary action of liquid passing through a winding path within the structure of the wick.

Various advantageous design characteristics may include, among other characteristics, their ability to efficiently transport liquid in a controlled manner by means of capillary action, their retention within the reservoir to prevent removal of the wick and to prevent access to the liquid contained therein and maintain their structural integrity and resistance to breakage or deformation during manufacture and use. It is also generally beneficial that liquid be retained in the wicking material. For example, it is desirable that the liquid be prevented from being drained under the action of gravity, such as when the reservoir is inverted.

However, many wicks currently available do not exhibit any number of these characteristics. For example, wicks made of fibrous, non-woven materials may permit liquid leakage under the action of gravity when the liquid reservoir is inverted. In addition, wicks made of fabric or non-woven materials tend to be mechanically weak and can be easily distorted or even disintegrated. Graphite or ceramic wicks can provide satisfactory leakage retention; however, these materials generally tend to be brittle and can fracture under stress. It is therefore desirable to identify an effective wicking material that offers advantages over existing materials at affordable costs.

### SUMMARY OF THE INVENTION

The present invention relates to the use of porous materials, for example, polymeric wicking materials, for transporting liquids from a reservoir in a vapor-dispensing device which addresses many of the shortcomings of the prior art. As described in additional detail below, the pore sizes and void volume ratios of the various wicking materials used in accordance with the present invention are selected to fall within a desired range to obtain effective control of liquid delivery. For example, in accordance with various aspects of the present invention, a porous wick material is comprised of various materials having pore sizes less than about 250 microns and void volume ratios on the order from about 25 to about 60%.

Additionally, the selection of certain materials, such as various polymeric materials, can provide additional characteristics such as resistance to fracturing and disintegration during uses, reduced leakage, and the ability to be configured in more shapes and sizes.

The wick materials in accordance with the present invention are useful as a transport mechanism for volatizing liquids and particularly, oily liquids (e.g., perfume) from vapor dispensing devices, such as an air freshener device. Nearly any conventional volatizable material, but especially volatizable fragrance materials, such as volatile odorous substances including essential oils, aromatic chemicals and the like, are suitable for use with the present invention as may other vaporizable materials. That is, a wide variety of fragrance materials as are now known to or hereafter devised by those skilled in the art of perfumery may be used in connection with the wicks of the present invention. These materials may comprise one or more natural materials, synthetic aromatic chemicals, and/or a mixture of both.

Further still, wicks made in accordance with the present invention can be designed to conform to various dimensions and shapes that allow for a variety of functional as well as aesthetic surface design features. That is, another advantage of wicks in accordance with various aspects of the present invention over the prior art, is their ability to be molded into a variety of shapes. Prior art wicks generally have been limited by their manufacturing processes to cylindrical shapes having a substantially uniform diameter over the length of the wick.

In accordance with these and other aspects of the present invention, described in greater detail below, the ease of application and performance of a liquid vapor dispenser is improved, resulting in greater consistency of product performance and reduced consumer frustration.

### BRIEF DESCRIPTION OF THE DRAWING FIGURE

A more complete understanding of the present invention may be derived by referring to the detailed description and claims when considered in connection with the figures, where like reference numbers refer to similar elements throughout the figures, and:

FIG. 1 is a liquid dispenser with a wick in accordance with an exemplary embodiment of the present invention;

FIG. 2 is a graph illustrating the results of fragrance delivery for an exemplary embodiment of the present invention; and

FIG. 3 is a graph illustrating liquid fragrance delivery data for wicks in accordance with the present invention as a function of time.

### DETAILED DESCRIPTION OF EXEMPLARY EMBODIMENTS

Additional aspects of the present invention will become evident upon review of the non-limiting exemplary embodiments described in the following specification taken in conjunction with the accompanying figures and tables provided.

As an exemplary embodiment, the wicking system of the present invention can be applied to liquid electric air fresheners such as those described in U.S. Pat. No. 6,104,867 issued Aug. 15, 2000 to Stathakis et al; U.S. Pat. No. 5,647,053 issued Jul. 8, 1997 to Schroeder et al; and U.S. Pat. No. 5,038,394 issued Aug. 6, 1991 to Hasegawa et al. Such air freshener devices generally include a thermal element or heating jacket that surrounds a wick. Elevation of the wick's temperature generally increases the kinetic rate of capillary transport of the reservoir liquid through the wick with sufficient speed to support accelerated vaporization at the wick's exterior surface.

The term “wick,” as used in accordance with the present invention, refers to the element used to transport the liquid to be dispensed, which typically will include some material, as well as the space created by pores contained therein. As used herein, “pores” refers to the cavities formed within the wick material itself. As will be discussed in greater detail below, “pore size” is used to describe the average diameter of a sample of pores of the wick material, and is expressed in microns. Also described in greater detail below, “void volume ratio” refers to the ratio of the volume of all the pores of the wick material to the overall volume of the wick itself (pores and wick material), and is generally expressed herein as a percentage.

Additionally, porous wicks in accordance with the present invention may be comprised of many materials now known or as yet unknown in the art. Specifically, any material which may be suitably configured to exhibit acceptable porosity, as will be described in greater detail herein, may be used. However, in the presently described non-limiting embodiment include various polymeric materials such as, ultra high molecular weight polyethylene, which generally have molecular weight, ranging from about 10,000 to about 100,000, high density polyethylene, polyvinylidene fluoride, nylon-6, polyethylenesulfone, polytetrafluoroethylene or other polymeric materials and mixtures thereof. Of these polymers, ultra high molecular weight polyethylene and high-density polyethylene exhibit suitable performance characteristics. Ultra high molecular weight polyethylene offers substantially improved performance because its material properties provide for appropriate transport kinetics as well as exhibiting suitable mechanical, chemical and thermodynamic stability.

That being said, various aspects of the present invention relate to the use of porous materials, preferably, porous polymeric materials as wicking materials for transporting liquids from a reservoir in a vapor-dispensing device. The pore sizes of the various types of wicking materials used in accordance with the present invention are suitably selected to obtain effective control of liquid delivery. Similarly, void volume ratios of wick also are suitably selected to obtain effective control of delivery of the liquid to the air and structural integrity. For example, as mentioned above, wicks in accordance with various aspects of the present invention preferably include porous polymeric wicks having pore sizes less than about 250 microns and void volume ratios from about 25 to about 60%, thus effectively obtaining properties comparable to various prior art materials, without necessarily sacrificing other desirable characteristics.

For example, wicks made of materials having substantially larger pore sizes may have a higher tendency to leak upon inversion of the reservoir, and also may tend to have less capacity for capillary transport of the liquid from the reservoir. On the other hand, wicks made of materials having substantially smaller pore sizes, while tending to be more resistant to leakage, often tend to wick liquids into the air too slowly, or not at all, resulting in poor transport kinetics.

Similarly, wick materials exhibiting void volume ratios above a suitable range may be too soft and flexible to perform as desired and may exhibit leakage. On the other hand, if the wick materials have void volume ratios below a suitable range, the rate of liquid transport through the wick may be lower than desired.

Wicking materials in accordance with the present invention are also advantageously selected to minimize clogging. That is, some prior art wicks tend to clog when operated for prolonged periods of time. For example, in some instances, when the wick materials are heated, the solvent component of the reservoir solution may be preferentially vaporized, thereby gradually concentrating viscous fragrance components within the wick matrix. This in turn can lead to the formation of resinous solids in the wick body and carbonization of the same. The aggregate effect results in clogging of the wick. Subsequent capillary transport of the reservoir solution will thereafter be substantially inhibited resulting in the failure of the vapor-dispensing device to perform efficiently, if at all, over a prolonged period of use. By suitable selection of pore size and void volume ratio, such clogging can be effectively minimized.

Further still, porous wicks in accordance with the present invention provide effective wicking properties sufficient enough that in heated vaporizers, the wick need not necessarily be placed in close proximity to the heating element of the device. Stated otherwise, because of the performance of wicks in accordance with the present invention, they can be placed further away from the heating element.

Generally, wick materials, in accordance with various aspects of the present invention, have pores with substantially the same spherical geometry and the pore size is the diameter of the largest cross-section for any particular pore space. For example, porous polymeric wick materials such as those provided by Porex Porous Products Group generally have pore sizes which do not vary by more than about 15% from a mean size.

Determining average pore size can be done by any member of means known or as yet unknown in the art. For example, various measuring instruments exist which are capable of accurately measuring pore size. For example, one instrument used to measure pore size and pore volume is the Mercury Intrusion Poresimeter. To measure pore size, the Poresimeter immerses the wick material with liquid mercury under pressure filling the pores and allowing measurement of the volume of mercury absorbed by the pores, and the total pore volume (v.) can be determined based on the volume of mercury. As more mercury fills in the pores of the wick, the pressure increases. The pressure profile is associated with the average porex size (p.) by the following relation:

$${P}_{s} = ( \frac{{V}_{p}}{{P}_{A}} )$$

While average pore size can be determined in any number of ways, in general, in accordance with various aspects of the present invention, average pore size of the various wick materials is suitably selected to ensure effective liquid delivery characteristics. As such, variations on average pore size may exist within particular wick materials and necessarily be dependent on the testing methodologies used. Average pre-size distributions will nevertheless generally be on the order of not more than 15%. As mentioned above, void volume ratio (Vᵥ) is the ratio of the volume of the pores of the wick material (Vₚ) including those pores that are interconnected to the surface of the wick as well as those that are sealed off by natural containment within the wick material to the total volume of the wick itself (V_{w}) or:

$${V}_{v} = ( \frac{{V}_{p}}{{V}_{w}} )$$

Any number of factors may dictate the void volume ratio, including the pore sizes and shapes and/or the uniformity of the sizes of the pores. In general, the materials selected for use in making the wicks in accordance with the present invention, as well as manufacturing techniques so utilized preferably result in a substantially uniform distribution of pores of substantially uniform size and volume throughout the wick matrix.

The total volume of the wick itself can be determined any number of ways, including by displacement or geometric equations. For example, for a typical cylindrical wick, V_{w} can be determined by the relationship:

$${V}_{w} = π ( \frac{d}{2} ) 2 {L}_{8}$$

where d is the outer diameter of the wick and L is the length of the wick.

The total volume of the pores of the wick can be determined by any number of ways as well. For example, the Mercury Instrusion Poresimeter mentioned above may be used. Alternatively, for example, for many materials used with wicks of the present invention, the density of the wick material is known. Density (δ) is generally expressed as a ratio of mass to volume. Thus, the volume of the wick material (Vₘ) can be determined by weighing the wick itself to determine its mass (m) and dividing the mass by the density of the material, or:

$${V}_{m} = \frac{m}{δ}$$

The void volume ratio (Vᵥ) is thus given by:

$${V}_{v} = ( 1 - \frac{{V}_{m}}{{V}_{w}} ) or$$

$${V}_{v} = ( \frac{{V}_{w} - {V}_{m}}{{V}_{w}} )$$

Preferably, the void volume used on connection with the various embodiments of the subject invention is derived in the data measured by the Mercury Intrusion Porosimeter approach, as discussed hereinabove.

As mentioned above, in accordance with various aspects of the present invention, pore sizes and void volume ratios are suitably selected to render a wick material for effective delivery of liquid materials. For example, in accordance with one preferred embodiment, pore sizes for effective wick performance are selected to be on the order of less than about 250 microns and the void volume ratio is selected to be on the order of less than about 60%. More preferably, in accordance with various aspects of the present invention, wick materials are suitably selected and configured to yield wicks having pore sizes in the range of from about 4 to about 40 microns, while the void volume ratio of such material is in the range from about 30% to about 40%.

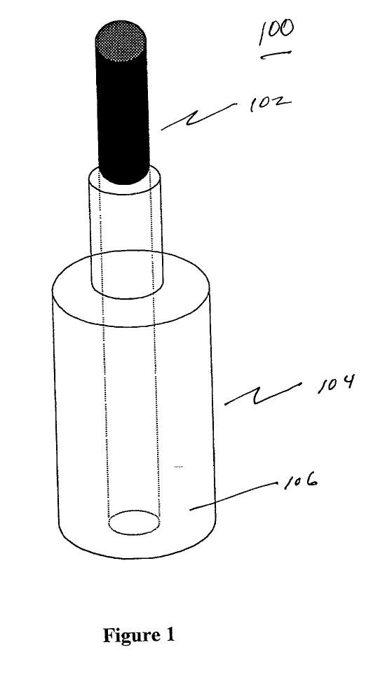

Selection of wicks with certain pore sizes and void volume ratios within such ranges may also prevent or reduce fragrance leakage and/or provide other advantages, such as advantages in the wicking rate. For example, in accordance with an exemplary embodiment of the present invention, FIG. 1 illustrates a simple vapor-dispensing test device 100 employing a porous polymer wick 102 in accordance with the present invention. Generally, dispensers 100 comprise wick 102, a reservoir 104 (or other bottle) and a fragrance oil 106 contained in reservoir 104. In this embodiment, wick 102 comprised a high molecular weight polyethylene having a pore size of 28 microns and a void volume ratio of 30%. Table 1, provided below, sets forth delivery rate data as determined by measuring the weight of volatized fragrance liquid by difference as a function of time.

TABLE 1

| Time | Weight of fragrance delivered |
|---|---|
| (hours) | (grams) |
| 3 | 0.07 |
| 18 | 0.21 |
| 27 | 0.27 |
| 42 | 0.34 |
| 69 | 0.46 |
| 163 | 0.78 |
| 213 | 0.93 |
| 241 | 1 |
| 307 | 1.1 |

As shown in Table 1, fragrance can be generally uniformly delivered over significant periods of time.

Within the pore size range of about 4.5 to about 29.0 microns and void volume ratios in the range of about 30 to about 35.1%, three porous polymer wicks, made in accordance the present invention, from high molecular weight and/or high density polyethylene, were found to have increased performance characteristics. Table 2, set forth below, shows the pore sizes and void volumes of these wicks. As set forth in the following example, wicks in accordance with the present invention exhibit liquid delivery generally comparable to that of graphite wicks having substantially similar dimensions.

TABLE 2

| Wick | | |
|---|---|---|
| sample | Pore size (microns) | Void volume (%) |
| A | 4.7 | 31.4 |
| B | 10.2 | 30.0 |
| C | 28.6 | 35.1 |

### EXAMPLE 1

Various wick materials in accordance with various aspects of the present invention have been prepared and the fragrance delivery of such wicks was compared to conventional fiber or graphite materials. Each of the wicks prepared from high molecular weight and/or high density polyethylene, namely ultra high molecular weight polyethylene (UHMW PE). Each of the wicks were configured to have a cross sectional diameter on the order of 7.24 mm and a length on the order of 66 mm. Each of the inventive wicks (denoted as A-1, A-2, B-1, B-2, C-1 and C-2 in the following Table 3) were selected to have the pore size and void volume ratios of wick samples A, B, C as forth above in Table 2. (For purposes of clarity, samples A-1 and A-2 each were configured to have pore sizes on the order of 4.6 microns and a void volume ration on the order of 31.4%, and so on for samples B-1, B-2, C-1 and C-2, in each corresponding to the B and C designations in Table 2).

Comparative graphite and polyester fiber wicks were also obtained. The graphite wicks were Earth Chemical Company, Ltd. wicks and the polyester fiber wicks were supplied from Porex Corporation. The comparative wicks were similarly dimensioned, i. e., having cross sectional diameters on the order of 7.24 mm and lengths on the order of 66 mm.

Each of these wicks were tested with liquid electric air freshener devices of the type Renuzit One Touch™ provided by The Dial Corp. operating substantially continuously at about 60-75° C. The fragrance delivery results are reported in Table 3 below. As illustrated, wicks in accordance with various aspects of the present invention exhibit liquid delivery rates generally comparable to that of graphite or fiber wicks having substantially similar dimensions.

TABLE 3

| | Weight of | | Weight of | | Weight of | | Weight of | |
|---|---|---|---|---|---|---|---|---|
| | fragrance | | fragrance | | fragrance | | fragrance | |
| Wick | delivered | Time | delivered | Time | delivered | Time | delivered | Time |
| Type | (grams) | (hours) | (grams) | (hours) | (grams) | (hours) | (grams) | (hours) |
| Porous | 4.25 | 72 | 7.28 | 141.1 | 10.08 | 188.35 | NA | 356.25 |
| Plastic | | | | | | | | |
| Sample | | | | | | | | |
| A-1 | | | | | | | | |
| Porous | 4.97 | 72 | 8.49 | 141.1 | 11.92 | 188.35 | 18.00 | 356.25 |
| Plastic | | | | | | | | |
| Sample | | | | | | | | |
| A-2 | | | | | | | | |
| Porous | 4.93 | 72 | 8.52 | 141.1 | 11.90 | 188.35 | 21.32 | 356.25 |
| Plastic | | | | | | | | |
| Sample | | | | | | | | |
| B-1 | | | | | | | | |
| Porous | 5.41 | 72 | 9.52 | 141.1 | 13.53 | 188.35 | 20.96 | 356.25 |
| Plastic | | | | | | | | |
| Sample | | | | | | | | |
| B-2 | | | | | | | | |
| Porous | 4.60 | 72 | 8.02 | 141.1 | 11.21 | 188.35 | 18.84 | 356.25 |
| Plastic | | | | | | | | |
| Sample | | | | | | | | |
| C-1 | | | | | | | | |
| Porous | 4.94 | 72 | 8.57 | 141.1 | 12.07 | 188.35 | 20.52 | 356.25 |
| Plastic | | | | | | | | |
| Sample | | | | | | | | |
| C-2 | | | | | | | | |
| Graphite | 4.27 | 72 | 7.67 | 141.1 | 10.97 | 188.35 | 20.21 | 356.25 |
| Sample 1 | | | | | | | | |
| Graphite | 4.98 | 72 | 9.08 | 141.1 | 13.06 | 188.35 | 22.72 | 356.25 |
| Sample 2 | | | | | | | | |
| Fiber | 3.65 | 72 | 6.62 | 141.1 | 10.02 | 188.35 | 22.89 | 356.25 |
| Sample 1 | | | | | | | | |
| Fiber | 1.98 | 72 | 4.23 | 141.1 | 6.10 | 188.35 | 14.41 | 356.25 |
| Sample 2 | | | | | | | | |
| Fiber | 1.28 | 72 | 3.85 | 141.1 | 6.82 | 188.35 | 15.97 | 356.25 |
| Sample 3 | | | | | | | | |
| Fiber | 2.10 | 72 | 4.60 | 141.1 | 7.25 | 188.35 | 15.78 | 356.25 |
| Sample 4 | | | | | | | | |

For example, Table 3 shows the pore sizes and void volume ratios of wicks having a rate of liquid delivery generally comparable to that of graphite or fiber wicks having substantially similar dimensions. Two samples for each wick were tested with liquid electric air freshener devices operating substantially continuously at about 25° C. ambient temperature. In these embodiments, the cross-sectional diameters of the wicks were about 7.24 mm with wick lengths of approximately 66 mm.

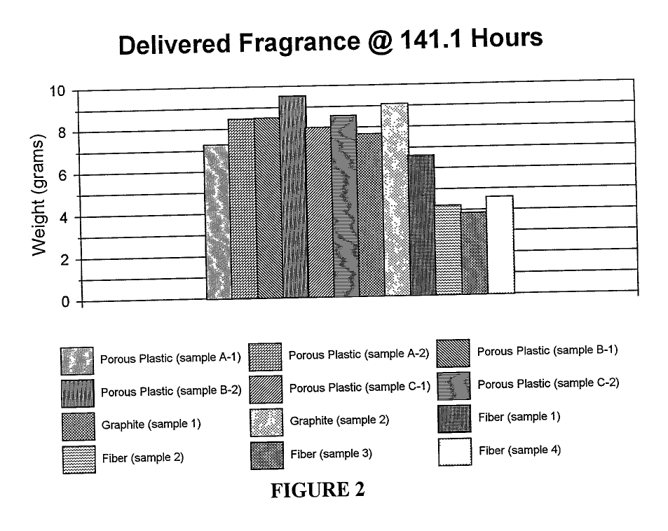

With reference now to FIG. 2 the results of fragrance delivery for various wicks further demonstrates that fragrance delivery results achieved over a 141.1 hour period compare favorably with conventional wick materials like fabric and graphite. For example, as clearly illustrated in FIG. 2, each of samples A-1, A-2, B-1, B-2, C-1 and C-2 each exhibited superior delivery rates than the comparative graphite wicks. In the case of inventive sample B-2, superior performance as compared to the comparative graphite wick (Sample 2) was observed. Additionally, beneficially, the delivery was also achieved without clogging, dripping or leaking.

As briefly noted above, in accordance with various aspects of the present invention, suitable wick materials are advantageously configured, such as through the selective of suitable pore sizes and/or void volume ratios to yield wicks which are effective to deliver liquids, e.g., fragrance materials, at rates comparable to conventional wick materials.

In accordance with various aspects of the present invention, the pore size is suitably selected to be on the order of from about 2 to about 250 microns, and more preferably in the range of from about 2 to about 70 microns. In certain applications, smaller pore size ranges may advantageously be selected, for example, such that the pore size is on the order of between about 3 to about 30 microns, and more preferably on the order of about 4 to about 5 microns to about 28 to about 30 microns. The standard deviation on the pore size distribution should be less than +/−20%, preferably +/−15%, and most preferably +/−6% of the average pore size.

In accordance with various other aspects of the present invention, the void volume ratio of the wick materials is suitably selected to be in the range of about 20 to about 60%, and more preferably in the range of from about 25 to about 45%. However, in certain applications more material may be effectively utilized and void volume ratios in the range of about 30 to about 40%, and more preferably in the range of about 31.5 to about 35% can be effectively employed.

In general, suitable selection of these characteristics, to wit, pore size and void volume ratio can be made depending upon the particular desired wick application. For example, in some cases, small pore sizes may be suitably selected to be combined with large void volume ratios, i.e., indicating a significant number of pores over a unit volume. Any number of combinations of pore size and void volume ratio may be selected so long as the resultant wick material is capable of providing substantially effective fluid delivery. However, in some cases, particularly in cases where the wick material comprises high density polyethylene (HDPE) and the wick is manufactured in accordance with conventional porous plastic processing techniques, pore sizes on the order of from about 25 to 30 microns and a void volume ratio on the order of between about 30 to about 40% have been found to enable the formation of a particularly effective wick material.

Moreover, of substantial benefit, polymer wicks in accordance with the present invention tend to exhibit various other advantageous properties. For example, such wicks tend to be generally more flexible and less brittle. Additionally, polymer wicks in accordance with the present invention provide generally more consistent and substantially quicker fragrance delivery when compared with fiber wicks. The mechanical strengths of the polymer wicks also tend to be generally greater than those of fiber wicks.

The distribution of pore sizes and void volume ratios within the inventive wicks may also be suitably selected, depending on particular applications, to exhibit various levels of apparent (also referred to as “effective”, or “net”) porosity. That is the portion of void space that excludes the sealed-off pores; can be minimized, while the formation of effectively interconnected pores which are accessible to the surface of the wick, are advantageously selected. Such selections may be a factor of the kinetic rate of capillary transport of fluids through the porous polymer material. Depending on the type of close-packing of the polymerized material, porosity can be selected in certain cases to be substantial.

Further, porous wicks in accordance with the present invention may also provide for both isotropic and anisotropic distributions of pore geometries and sizes throughout the wick matrix, thereby tending to substantially improve the capillary transport properties of same.

Optionally, the pore size and void volume ratio of the various wicks in accordance with various aspects of the present invention may be suitably selected to enhance the anti-leaking properties of the wicks of the present invention, as set forth in the following Example 2.

### Example 2

Various porous plastic wick materials were prepared with varying pore sizes, substantially along the lines as set forth in Example 1, but having the pore sizes specified in Table 4, below. In each case, void volume ratios were on the order of about 30 to about 40%. The anti-leaking properties of these inventive wicks were compared with graphite and fiber wicks having the general properties also specified in Table 4.

In order to test the transport capability and capacity of wicks in accordance with the present invention, the time for fragrance to travel approximately 66 mm and the weight of fragrance absorbed by the wick over that time were measured. To test the anti-leaking properties, the fragrance-reservoir was inverted to allow fragrance to flow toward the fitment-neck under the action of gravity. The results are shown in Table 4 below.

TABLE 4

| | | Weight of | | |
|---|---|---|---|---|
| | | perfume | Time to travel | |
| | Pore size | absorbed | 66 mm | Inverted |
| Wick type | (microns) | (grams) | (minutes:seconds) | leakage |
| Porous Plastic | 20 | 1.01 | 3:40 | No |
| Porous Plastic | 15 | 0.67 | 8:09 | No |
| Porous Plastic | 7 | 0.74 | 55:00 | No |
| Graphite | 5 | 0.16 | 1440:00 | No |
| Fiber Rod SU2 | NA | NA | * | Yes |
| Fiber Rod SU14 | NA | NA | * | Yes |
| Fiber Rod SU44 | NA | 1.33 | 2:06 | Yes |
| Fiber Rod SU53 | NA | 1.27 | 2:50 | Yes |

* Wicking stopped short of the 66 mm distance

The results show that porous polymer wicks in accordance with the present invention generally provide higher levels of liquid reservoir retention and fragrance wicking rate than those of graphite wicks. Such features achieve substantially improved and consistent liquid fragrance delivery performance in a vapor-dispensing device. Additionally, where fiber wicks permit liquid to drain out upon inversion of the reservoir, porous polymer wicks in accordance with the present invention, particularly, those having pore sizes of about 5-30 microns, showed no substantial fluid leakage upon inversion.

When the wick materials selected comprise suitable materials, for example, polymeric materials, the wicks formed in accordance with the present invention may also advantageously be configured to exhibit various different shapes. In this regard, reference is made to our pending application, entitled “Method and Apparatus for Fastening a Fluid Transport Mechanism to a Container” filed on Oct. 9, 2001, U.S. Ser. No.______, the subject matter of which is hereby incorporated herein by reference.

Finally, while various principles of the present invention have been described by way of the exemplary embodiments described herein, these and other combinations and/or modifications of the above-described structures, arrangements, proportions, elements, materials or components used in the practice of the present invention, in addition to those not specifically recited, may be varied or otherwise particularly adapted by those skilled in the art without departing from the general principles of the same.

We claim:

1. A wick material, comprising a porous material, said porous material having pores less than about 250 microns and a void volume ratio of less than about 60%.

2. A wick material in accordance with claim 1, wherein said porous material is a high density polyethylene.

3. A porous polymeric wick for use in connection with an air freshening device consisting essentially of:
a high density polyethylene wicking material having a void
volume ratio of from about 25 to about 60% and having an
average pore size on the order of less than 250 microns.

4. The wick of claim 3 wherein said wicking material has an average pore size in the range of from about 2 to about 70 microns.

5. The wick of claim 4 wherein said wicking material has an average pore size in the range of from about 3 to about 30 microns.

6. The wick of claim 5 wherein said wicking material has an average pore size in the range of from about 4 to about 28 microns.

7. The wick of claim X wherein said wicking material has an average pore size of about 30 microns.

8. The wick of claim 6 wherein said void volume ratio is in the range of from about 30 to about 40%.

9. A vapor dispensing device comprising:
a reservoir containing a volatizable liquid
a wicking material in fluid communication with said
volatizable liquid
said vapor dispensing device improved wherein said
wicking material exhibits an average pore size in the range
of about 4.5 to 29.0 microns and a void volume ratio on the
order of between about 30 to about 35%.

10. The device of claim 3, further comprising a vaporizing device configured to effect vaporization of the volatizable liquid contained on said wicking material.
